(19) United States
(12) Patent Application Publication
(10) Pub. No.: US 2024/0245385 A1
(43) Pub. Date: Jul. 25, 2024

(54) CLICK-TO-CORRECT FOR AUTOMATIC VESSEL LUMEN BORDER TRACING

(71) Applicant: Boston Scientific Scimed Inc., Maple Grove, MN (US)

(72) Inventor: Marcos Zachary, Inver Grove Heights, MN (US)

(73) Assignee: Boston Scientific Scimed Inc., Maple Grove, MN (US)

(21) Appl. No.: 18/411,774

(22) Filed: Jan. 12, 2024

Related U.S. Application Data
(60) Provisional application No. 63/439,985, filed on Jan. 19, 2023.

(51) Int. Cl. A61B 8/08; A61B 8/00; A61B 8/12; G06T 7/11

(52) U.S. Cl. CPC A61B 8/085; A61B 8/0891; A61B 8/12; A61B 8/465; G06T 7/11; G06T 2207/20084

(57) ABSTRACT

The present disclosure provides a system and technique to correct a lumen border based on identified region(s) on a IVUS image. The identified region(s) are received via an input device and indicate locations inside or outside the actual lumen border and further can indicate areas where an automatically identified lumen border is inaccurate. An updated lumen border is determined based on the region(s).

### CROSS-REFERENCE TO RELATED APPLICATIONS

This application claims the benefit of U.S. Provisional Patent Application Ser. No. 63/439,985 filed Jan. 19, 2023, the disclosure of which is incorporated herein by reference.

### TECHNICAL FIELD

The present disclosure generally relates to intravascular ultrasound (IVUS) imaging systems. Particularly, but not exclusively, the present disclosure relates to identifying a vascular border.

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

IVUS imaging systems includes a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

However, it can be difficult for physicians to identify anatomical segments, lumen (vessel) borders, disease presence, plaque burden, ultrasound artifacts, etc. from the raw IVUS images. Further, it is difficult to correlate raw IVUS images to angiogram and venogram images. Thus, there here is a need for user interfaces and software tools to communicate information from the raw IVUS images to a user.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides an improved lumen detection system in which areas of poor lumen detection are identified and the lumen is corrected in these areas automatically.

The disclosure can be implemented as a method, comprising: receiving, via an input device, an indication of a region of an intravascular ultrasound (IVUS) image of a vessel of a patient corresponding to a location either inside or outside a lumen border of the vessel; generating, at a processing component of an IVUS system, an updated lumen border based on an initially detected lumen border and the region; generating a graphical user interface (GUI) comprising visualizations of the IVUS image and the updated lumen border; and causing the GUI to be displayed on a display.

In further embodiments, the method can comprise receiving a series of IVUS images of the vessel of a patient from an IVUS catheter, the series of IVUS images comprising a plurality of frames, the IVUS image a one of the plurality of frames.

In further embodiments, the method can comprise automatically detecting the initially detected lumen border based on the IVUS image.

In further embodiments, the method can comprise generating an initial GUI comprising visualizations of the IVUS image and the initially detected lumen border; and causing the initial GUI to be displayed on a display.

In further embodiments of the method, receiving the indication of the region of the IVUS image of the vessel comprises receiving via a mouse or a touch screen an indication of a location on the GUI corresponding to the region.

In further embodiments of the method, the region comprises an indication of whether the region is within or without the lumen border.

In further embodiments of the method, generating the updated lumen border comprises deriving a graph cut segmentation of the IVUS image; and identifying the updated lumen border based on the graph cut segmentation and an iterative energy minimization where the region is the minimization parameter.

In further embodiments of the method, generating the updated lumen border comprises deriving a ranked list of a plurality of segmentation hypotheses; and identifying the updated lumen border based on the ranked list of the plurality of segmentation hypotheses and the region.

In further embodiments of the method, generating the updated lumen border comprises concatenating a Euclidian distance map of the IVUS image and the region to generate a pair of images; generating the updated lumen border based on an inference from a machine learning model using the pair of images as input to the machine learning model.

In further embodiments of the method, the machine learning model is a neural network.

In further embodiments of the method, the Euclidian distance map is based on a red, green, blue (RBG) channel segregation of the IVUS images and the region resulting in a plurality of pairs of images.

In further embodiments of the method, the region is a first region, and the method comprises receiving, via the input device, an indication of a second region of the IVUS image of the vessel of the patient corresponding to a location either within or without the lumen border of the vessel, wherein the Euclidian distance map is based on the first region and the second region resulting in a plurality of pairs of images.

In further embodiments of the method, the first region is inside the lumen border and the second region is outside the lumen border.

With some embodiments, the disclosure can be implemented as an apparatus, comprising a processor coupled to a memory, the memory comprising instructions executable by the processor, the processor configured to couple to an intravascular ultrasound (IVUS) imaging system and configured to execute the instructions, which instructions when executed cause the processor to implement the method of any embodiments described herein.

With some embodiments, the disclosure can be implemented as at least one machine readable storage device, comprising a plurality of instructions that in response to being executed by a processor of an intravascular ultrasound (IVUS) imaging system cause the processor to implement the method of any embodiments described herein.

In some embodiments, the disclosure can be implemented as an apparatus for an intravascular ultrasound (IVUS) imaging system, the apparatus can comprise a display; a computer input device; a processor coupled to the computer input device and the display; and a memory coupled to the processor, the memory comprising instructions executable by the processor, which when executed cause the processor to: receive, via the computer input device, an indication of a region of an IVUS image of a vessel of a patient corresponding to a location either inside or outside a lumen border of the vessel; generate an updated lumen border based on an initially detected lumen border and the region; generate a graphical user interface (GUI) comprising visualizations of the IVUS image and the updated lumen border; and cause the GUI to be displayed on the display.

In further embodiments of the apparatus, the region comprises an indication of whether the region is within or without the lumen border.

In further embodiments of the apparatus, the instructions, which when executed by the processor further cause the processor to derive a graph cut segmentation of the IVUS image; and identify the updated lumen border based on the graph cut segmentation and an iterative energy minimization where the region is the minimization parameter.

In further embodiments of the apparatus, the instructions, which when executed by the processor further cause the processor to derive a ranked list of a plurality of segmentation hypotheses; and identify the updated lumen border based on the ranked list of the plurality of segmentation hypotheses and the region.

In further embodiments of the apparatus, the instructions, which when executed by the processor further cause the processor to concatenate a Euclidian distance map of the IVUS image and the region to generate a pair of images; and generate the updated lumen border based on an inference from a machine learning model using the pair of images as input to the machine learning model.

With some embodiments, the disclosure can be implemented as at least one machine readable storage device, comprising a plurality of instructions that in response to being executed by a processor of an intravascular ultrasound (IVUS) imaging system cause the processor to receive, via the computer input device coupled to the processor, an indication of a region of an IVUS image of a vessel of a patient corresponding to a location either inside or outside a lumen border of the vessel; generate an updated lumen border based on an initially detected lumen border and the region; generate a graphical user interface (GUI) comprising visualizations of the IVUS image and the updated lumen border; and cause the GUI to be displayed on a display coupled to the processor.

In further embodiments of the storage device, the Euclidian distance map is based on a red, green, blue (RBG) channel segregation of the IVUS images and the region resulting in a plurality of pairs of images, and wherein the region is a first region, the instructions in response to be executed by the processor, further cause the processor to receive, via the input device, an indication of a second region of the IVUS image of the vessel of the patient corresponding to a location either within or without the lumen border of the vessel, wherein the Euclidian distance map is based on the first region and the second region resulting in a plurality of pairs of images.

In further embodiments of the storage device, the first region is inside the lumen border and the second region is outside the lumen border.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an IVUS imaging system in accordance with at least one embodiment.

FIG. 2 illustrates an image.

FIG. 3A and FIG. 3B illustrate IVUS images.

FIG. 4 illustrates an IVUS image visualization system in accordance with at least one embodiment.

FIG. 5A illustrates an IVUS image.

FIG. 5B illustrates the IVUS image of FIG. 5A with an automatically detected lumen boundary.

FIG. 5C illustrates the IVUS image of FIG. 5B with selected regions identified.

FIG. 5D illustrates the IVUS image of FIG. 5C with a corrected lumen boundary.

FIG. 5E illustrates the IVUS image of FIG. 5A with the corrected lumen boundary.

FIG. 6 illustrates a logic flow in accordance with at least one embodiment.

FIG. 7 illustrates a logic flow in accordance with at least one embodiment.

FIG. 8 illustrates a computer-readable storage medium.

FIG. 9 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVUS systems and automatic assessment of the IVUS images. In particular, the disclosure provides a graphical user interface (GUI) arranged to convey information related to the IVUS images and lesion assessment and provide for the user to manipulate the information. As such, an example IVUS imaging system, patient vessel, and series of IVUS images are described.

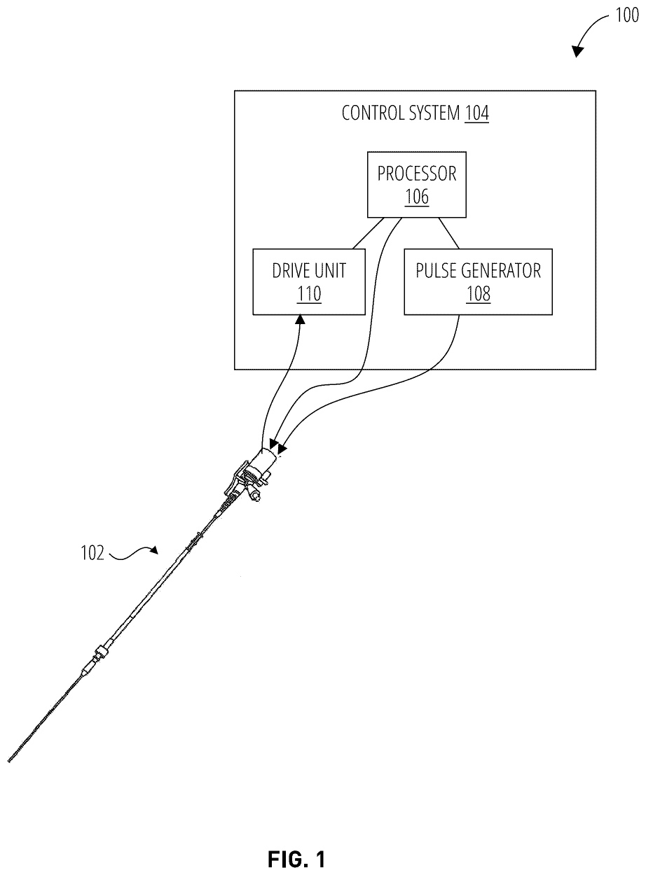

Suitable IVUS imaging systems include, but are not limited to, one or more transducers disposed on a distal end of a catheter configured and arranged for percutaneous insertion into a patient. FIG. 1 illustrates one embodiment of an IVUS imaging system 100. The IVUS imaging system 100 includes a catheter 102 that is couplable to a control system 104. The control system 104 may include, for example, a processor 106, a pulse generator 108, and a drive unit 110. The pulse generator 108 forms electric pulses that may be input to one or more transducers (not shown) disposed in the catheter 102.

With some embodiments, mechanical energy from the drive unit 110 can be used to drive an imaging core (also not shown) disposed in the catheter 102. In at least some embodiments, electric signals transmitted from the one or more transducers may be input to the processor 106 for processing. In at least some embodiments, the processed electric signals from the one or more transducers can be used to form a series of images, described in more detail below. For example, a scan converter can be used to map scan line samples (e.g., radial scan line samples, or the like) to a two-dimensional Cartesian grid, which can be used as the basis for a series of IVUS images that can be displayed for a user.

In at least some embodiments, the processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where IVUS imaging system 100 is configured for automatic pullback, the drive unit 110 can control the velocity and/or length of the pullback.

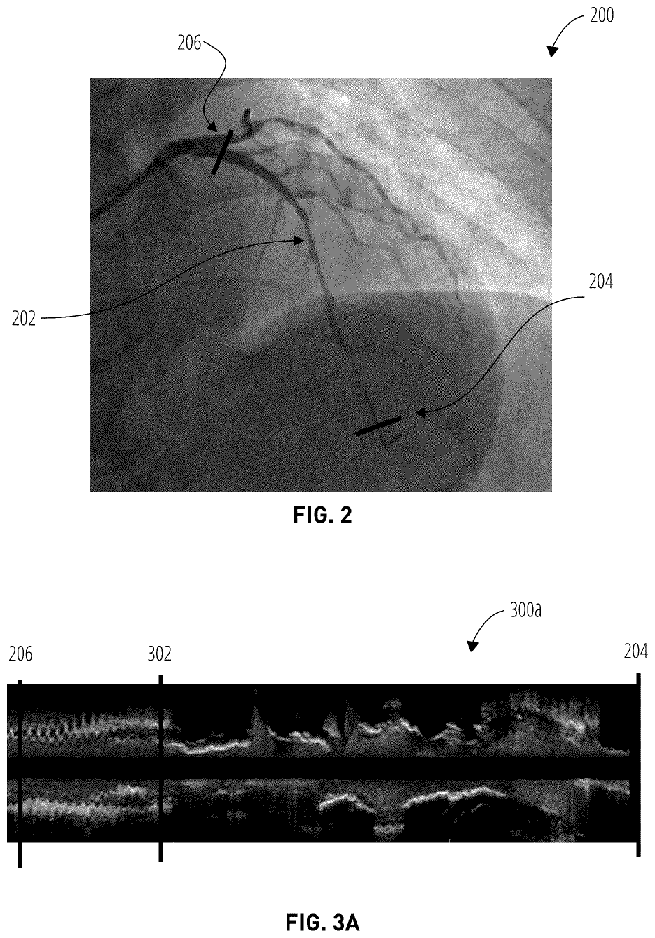

FIG. 2 illustrates an image 200 of a vessel 202 of a patient. As described, IVUS imaging systems (e.g., IVUS imaging system 100, or the like) are used to capture a series of images or a “recording” or a vessel, such as, vessel 202. For example, an IVUS catheter (e.g., catheter 102) is inserted into vessel 202 and a recording, or a series of IVUS images, is captured as the catheter 102 is pulled back from a distal end 204 to a proximal end 206. The catheter 102 can be pulled back manually or automatically (e.g., under control of drive unit 110, or the like).

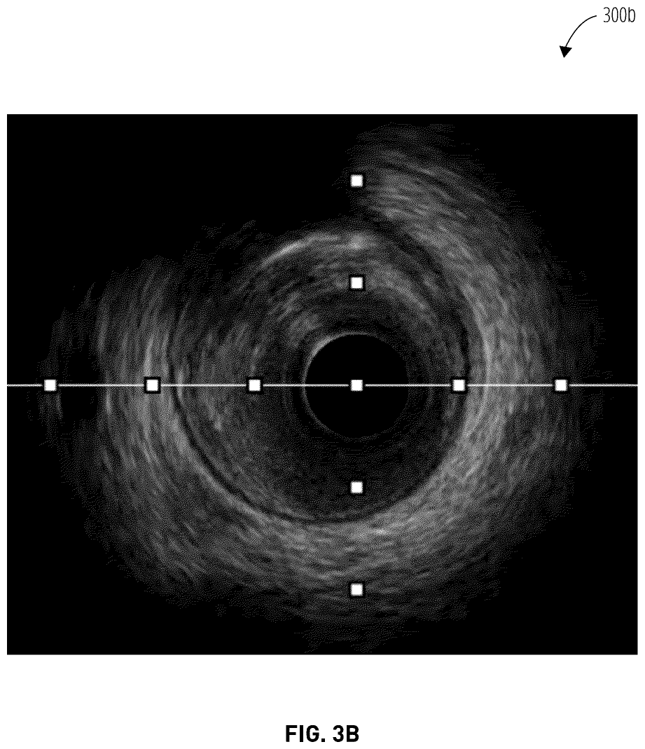

FIG. 3A and FIG. 3B illustrates two-dimensional (2D) representations of IVUS images of vessel 202. For example, FIG. 3A illustrates IVUS images 300a depicting a longitudinal view of the IVUS recording of vessel 202 between proximal end 206 and distal end 204.

FIG. 3B illustrates an image frame 300b depicting an on-axis (or short axis, or cross-section) view of vessel 202 at point 302. Said differently, image frame 300b is a single frame or single image from a series of IVUS images that can be captured between distal end 204 and proximal end 206 as described herein. As introduced above, the present disclosure provides systems and techniques to process raw IVUS images to detect lumen (or vessel) borders. In particular, the present disclosure provides a technique to correct or improve automatic detection of lumen borders.

For example, IVUS images 300a depicts an entire series of IVUS images taken of vessel 202 between distal end 204 and proximal end 206. At specific locations along the vessel 202, a physician may desire to look at the on-axis view of the vessel 202 to assess the health of the vessel. With some IVUS imaging systems (e.g., IVUS imaging system 100, or the like) a vessel border can be automatically drawn or indicated on an image of the on-axis view of the vessel (described in greater detail below). However, often the vessel border is incorrect or inaccurate. Conventional IVUS imaging systems require the physician to manually correct the vessel border, which is a time consuming and tedious process. The present disclosure provides to an automated technique to correct inaccurate vessel borders.

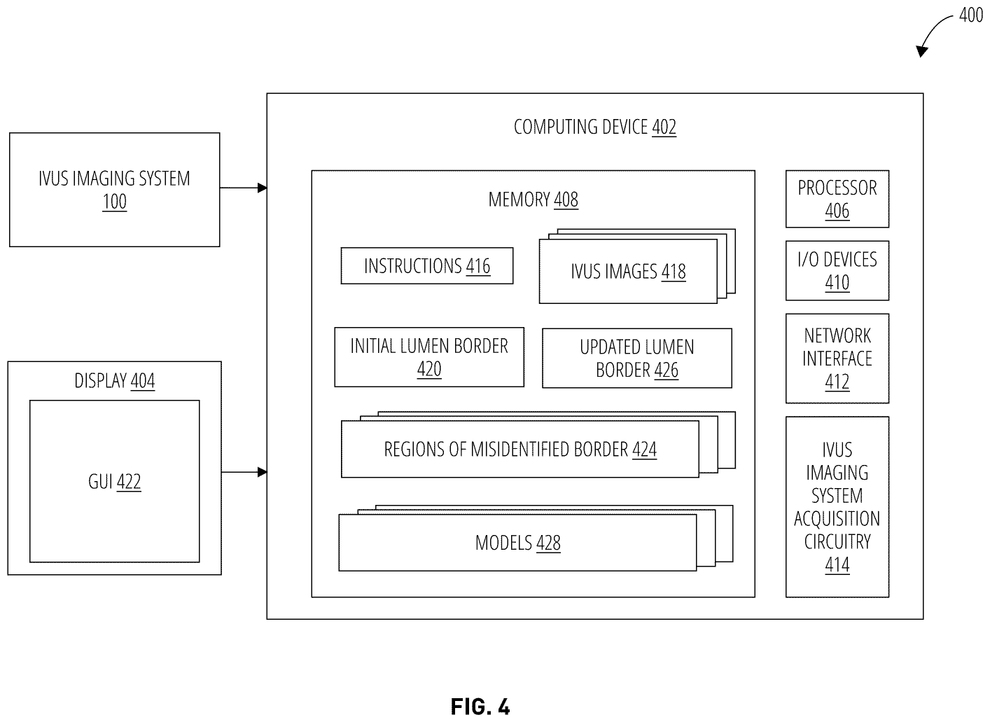

FIG. 4 illustrates an IVUS image visualization system 400, according to some embodiments of the present disclosure. In general, IVUS image visualization system 400 is a system for processing, annotating, and presenting IVUS images. IVUS image visualization system 400 can be implemented in a commercial IVUS guidance or navigation system, such as, for example, the AVVIGO ® Guidance System available from Boston Scientific ®. The present disclosure provides advantages over prior or conventional IVUS navigation systems in that the improved GUI will reduce the time needed for patients to be in treatment. For example, the present disclosure can be implemented in an IVUS navigation system to efficiently communicate IVUS information to a user and allow the user to manipulate the information.

With some embodiments, IVUS image visualization system 400 could be implemented as part of control system 104 of IVUS imaging system 100. Alternatively, control system 104 could be implemented as part of IVUS image visualization system 400. As depicted, IVUS image visualization system 400 includes a computing device 402. Optionally, IVUS image visualization system 400 includes IVUS imaging system 100 and display 404.

Computing device 402 can be any of a variety of computing devices. In some embodiments, computing device 402 can be incorporated into and/or implemented by a console of display 404. With some embodiments, computing device 402 can be a workstation or server communicatively coupled to IVUS imaging system 100 and/or display 404. With still other embodiments, computing device 402 can be provided by a cloud based computing device, such as, by a computing as a service system accessibly over a network (e.g., the Internet, an intranet, a wide area network, or the like). Computing device 402 can include processor 406, memory 408, input and/or output (I/O) devices 410, network interface 412, and IVUS imaging system acquisition circuitry 414.

The processor 406 may include circuity or processor logic, such as, for example, any of a variety of commercial processors. In some examples, processor 406 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 406 may include graphics processing portions and may include dedicated memory, multiple-threaded processing and/or some other parallel processing capability. In some examples, the processor 406 may be an application specific integrated circuit (ASIC) or a field programmable integrated circuit (FPGA).

The memory 408 may include logic, a portion of which includes arrays of integrated circuits, forming non-volatile memory to persistently store data or a combination of non-volatile memory and volatile memory. It is to be appreciated, that the memory 408 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 410 can be any of a variety of devices to receive input and/or provide output. For example, I/O devices 410 can include, a keyboard, a mouse, a joystick, a foot pedal, a display, a touch enabled display, a haptic feedback device, an LED, or the like.

Network interface 412 can include logic and/or features to support a communication interface. For example, network interface 412 may include one or more interfaces that operate according to various communication protocols or standards to communicate over direct or network communication links. Direct communications may occur via use of communication protocols or standards described in one or more industry standards (including progenies and variants). For example, network interface 412 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 412 can include logic and/or features to enable communication over a variety of wired or wireless network standards (e.g., 802.11 communication standards). For example, network interface 412 may be arranged to support wired communication protocols or standards, such as, Ethernet, or the like. As another example, network interface 412 may be arranged to support wireless communication protocols or standards, such as, for example, Wi-Fi, Bluetooth, ZigBee, LTE, 5G, or the like.

The IVUS imaging system acquisition circuitry 414 may include circuity including custom manufactured or specially programmed circuitry configured to receive or receive and send signals between IVUS imaging system 100 including indications of an IVUS run, a series of IVUS images, or a frame or frames of IVUS images.

Memory 408 can include instructions 416. During operation processor 406 can execute instructions 416 to cause computing device 402 to receive (e.g., from IVUS imaging system 100, or the like) a recording of an “IVUS run” and store the recording as IVUS images 418 in memory 408. For example, processor 406 can execute instructions 416 to receive information elements from IVUS imaging system 100 comprising indications of IVUS images captured by catheter 102 while being pulled back from distal end 204 to proximal end 206, which images can include indications of the anatomy and/or structure of vessel 202, including vessel walls and plaque. It is to be appreciated that IVUS images 418 can be stored in a variety of image formats or even non-image formats or data structures that comprise indications of vessel 202. Further, IVUS images 418 includes several “frames” or individual images that, when represented co-linearly can be used to form an image of the vessel 202, such as, for example, as represented by IVUS images 300a and/or 300b.

Processor 406 can execute instructions 416 to identify an initial lumen border 420 based on IVUS images 418 and generate a GUI 422 comprising a visual indication of the IVUS images 418 and initial lumen border 420. It is to be appreciated that a variety of different techniques exist for detecting lumen (e.g., vessel) borders. The present disclosure does not attempt to completely describe these systems and is instead, intended to provide systems and methods to correct inaccurate borders identified by such automated techniques. However, a general description of an automated lumen border detection process is provided for clarity of presentation. In such an illustrative process, processor 406 can execute instructions 416 to modulate IVUS images 418 (e.g., to identify different tissue types, different regions of tissue, border between tissue and air, etc.) and identify segments of the lumen border based on the modulated image data. Accordingly, initial lumen border 420 includes several border segments (or line segments). Said differently, initial lumen border 420 comprises several points on IVUS images 418, which when connected identify an outline of the predicted vessel border. Processor 406 can execute instructions 416 to generate GUI 422 comprising indications of IVUS images 418 and initial lumen border 420 and cause GUI 422 to be displayed on display 404.

As noted above, automated vessel border prediction methods are not always accurate. As such, a physician viewing GUI 422 may determine that initial lumen border 420 is inaccurate. To better assist the physician in treating the patient, a corrected outline or depiction of the vessel border may be desired. As such, processor 406 can execute instructions 416 to receive regions of misidentified border 424 and to generate an updated lumen border 426 based on IVUS images 418, initial lumen border 420, regions of misidentified border 424, and models 428. This is described in greater detail below with reference to example images of a vessel lumen.

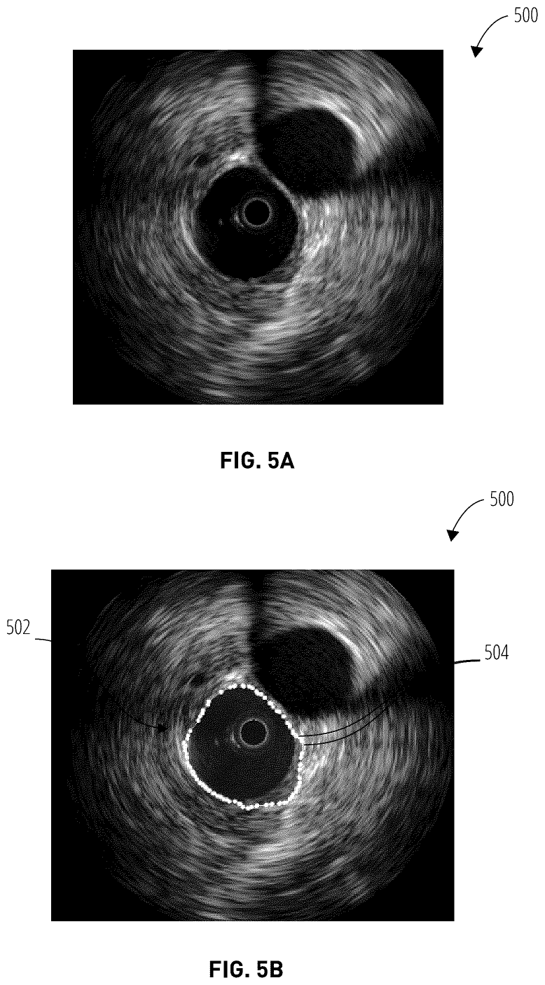

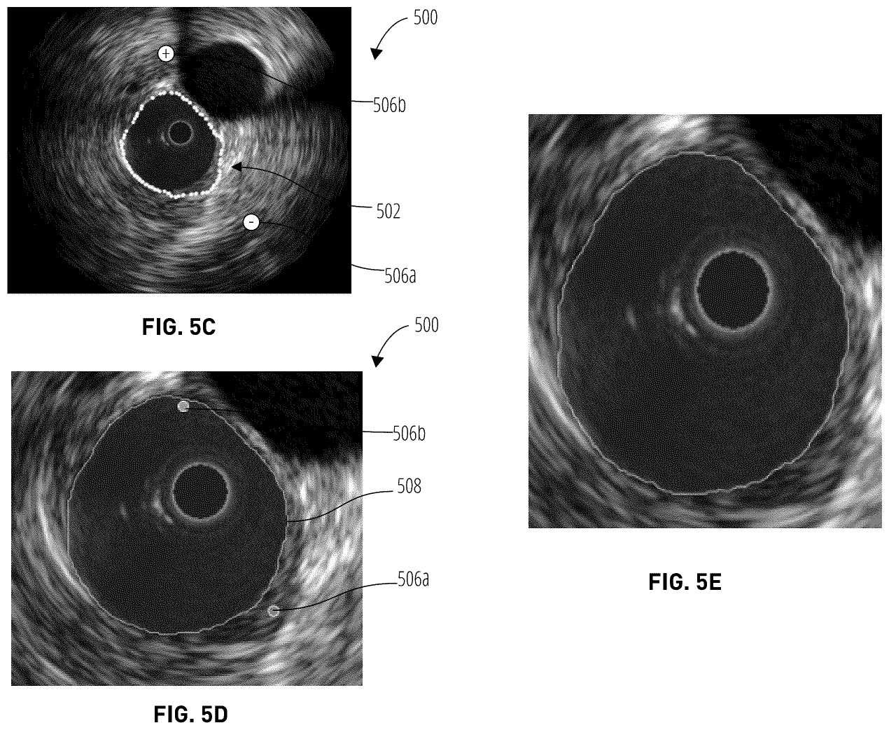

FIG. 5A illustrates an image 500 showing an on-axis (short axis, cross-section, etc.) view of a vessel. Image 500 can correspond to a frame of IVUS images 418. As noted above, processor 406 can execute instructions 416 to automatically detect (or identify) a border of the vessel depicted in IVUS images 418.

To that end, FIG. 5B illustrates image 500 showing the on-axis view of the vessel as well as an automatically detected lumen border 502. As can be seen, the automatically detected lumen border 502 comprises several points 504, which when connected for automatically detected lumen border 502. However, as further noted above, in some instances, automatically detected lumen border 502 is incorrect. As can be seen, automatically detected lumen border 502 is not correct in the image displayed in FIG. 5B. Conventionally, to correct inaccurate automatically detected lumen border 502, a physician would need to manually move each points 504. The present disclosure, however, provides that a physician can designate points on image 500 in which the border is inaccurate.

For example, FIG. 5C illustrates image 500 showing automatically detected lumen border 502 as well as points 506a and 506b. In general, points 506a and/or 506b can be designated as identifying a region within the lumen border (e.g., point 506b) or a region outside the lumen border (e.g., point 506a). With some embodiments, processor 406 can execute instructions 416 to receive indications (e.g., from a mouse, from a touch screen, from another input device, or the like) of points 506a and/or 506b as well as an indication of whether the points designate regions inside out outside the lumen border. It is to be appreciated that any number of points 506a and 506b can be received (e.g., one point, two points as shown, three points, four points, etc.). Examples are not limited in this context.

Responsive to receiving an indication of points 506a and/or 506b, an updated lumen border can be determined from the original lumen border, the image 500, and a model. For example, processor 406 can execute instructions 416 to determine updated lumen border 426 from IVUS images 418 (e.g., image 500), initial lumen border 420 (e.g., automatically detected lumen border 502), and regions of misidentified border 424 (e.g., points 506a and/or 506b). For example, FIG. 5D illustrates image 500 showing an updated lumen border 508 determined based on automatically detected lumen border 502, point 506a, point 506b, and image 500. As can be seen, the updated lumen border 508 includes points point 506b within the border and excludes points point 506a from within the border.

FIG. 5E illustrates image 500 showing just the updated lumen border 508. In some embodiments, responsive to determining updated lumen border 508, a GUI can be generated to depict the updated lumen border 508. For example, processor 406 can execute instructions 416 to generate GUI 422 comprising an indication of the frame of IVUS images 418 (e.g., image 500) and the updated lumen borders 426 (e.g., updated lumen border 508).

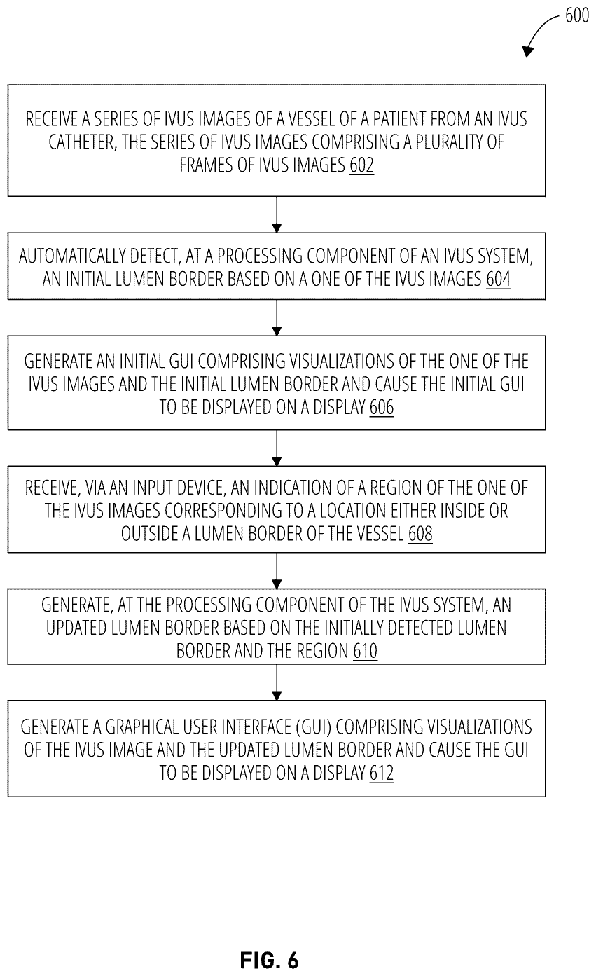

FIG. 6 illustrates a logic flow 600 to generate an updated lumen border, according to some embodiments of the present disclosure. The logic flow 600 can be implemented by IVUS image visualization system 400 and will be described with reference to IVUS image visualization system 400 for clarity of presentation. However, it is noted that logic flow 600 could also be implemented by an IVUS system different than IVUS image visualization system 400.

Logic flow 600 can begin at block 602. At block 602 “receive a series of intravascular ultrasound (IVUS) images of a vessel of a patient, the series of IVUS images comprising a plurality of frames” a series of IVUS images captured via an IVUS catheter percutaneously inserted in a vessel of a patent can be received. For example, information elements comprising indications of IVUS images 418 can be received from IVUS imaging system 100 where catheter 102 is (or was) percutaneously inserted into vessel 202. The IVUS images 418 can comprise frames of images representative of images captured while the catheter 102 is pulled back from distal end 204 to proximal end 206. Processor 406 can execute instructions 416 to receive information elements comprising indications of IVUS images 418 from IVUS imaging system 100, or directly from catheter 102 as may be the case.

Continuing to block 604 “automatically detect, at a processing component of an IVUS system, an initial lumen border based on a one of the IVUS images” an initial lumen border is automatically detected by processing circuitry of the IVUS system. For example, processor 406 can execute instructions 416 to automatically generate initial lumen border 420 from a one of the IVUS images received at block 602.

Continuing to block 606 “generate an initial GUI comprising visualizations of the one of the IVUS images and the initial lumen border and cause the initial GUI to be displayed on a display” an initial GUI comprising visualizations of the one (e.g., frame) of the IVUS image and the initially detected lumen border is generated. For example, processor 406 can execute instructions 416 to generate GUI 422 comprising an indication of IVUS images 418 (e.g., an on-axis view of the vessel) and initial lumen border 420.

Continuing to block 608 “receive, via an input device, an indication of a region of the one of the IVUS images corresponding to a location either inside or outside a lumen border of the vessel” an indication of a region of the IVUS image corresponding to a location either inside or outside the actual lumen border is received via an input device of the IVU system. For example, processor 406 can execute instructions 416 to receive (e.g., via input and/or output (I/O) devices 410 such as a mouse, track pad, pen style input device, touch screen, or the like) a region. (or regions) of the IVUS image and an indication of the whether the region is inside or outside the actual lumen border of the vessel.

Continuing to block 610 “generate, at the processing component of the IVUS system, an updated lumen border based on the initially detected lumen border and the region” an updated lumen border is generated from the initially detected lumen border and the region (or regions). For example, processor 406 can execute instructions 416 to generate updated lumen border 426 from regions of misidentified border 424 and initial lumen border 420. With some examples, processor 406 can execute instructions 416 to derive a graph cut segmentation of the IVUS image and determine the update lumen border based on applying an iterative energy minimization algorithm (e.g., models 428) to the graph cut segmentation using the region (or regions) as the minimization parameter.

In some embodiments, processor 406 can execute instructions 416 to derive a ranked list of a plurality of segmentation hypotheses of the one of the IVUS images 418 and use the regions of misidentified border 424 as a supervisory signal to identify new segments for the updated lumen border 426 based on a search algorithm (e.g., models 428).

With some embodiments, processor 406 can execute instructions 416 to concatenate a Euclidian distance map (e.g., RGB channel based map, or the like) of the one of the IVUS images 418 to generate a pair of images and can use the pairs of images as input to a machine leaning model (e.g., models 428) to generate the updated lumen border 426 from the model inference. In such embodiments, the model can be a neural network (NN), a fully convolutional network (FCN), a convoluted neural network (CNN), or the like.

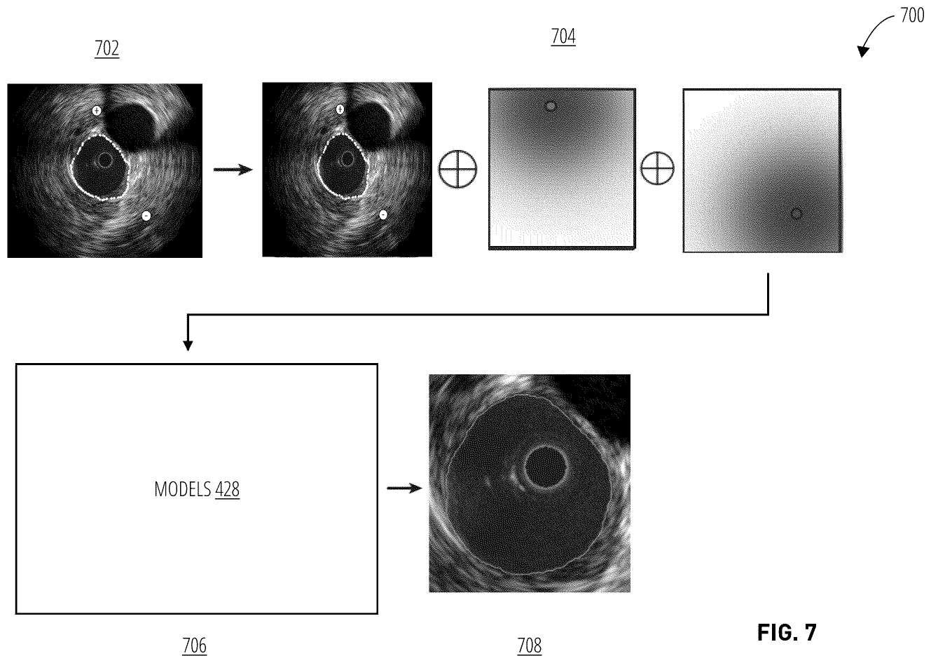

FIG. 7 illustrates a logic flow 700 to generate an updated border based on Euclidean distance and a machine learning model. With some embodiments, logic flow 700 can be implemented by an IVUS system (e.g., IVUS image visualization system 400) as part of logic flow 600. Logic flow 700 can begin at block 702 where regions of misidentified border 424 can be identified. As described above, the regions can indicate areas either inside or outside the actual lumen border. Continuing to block 704 the image and regions can be concatenated using Euclidean distance and channel maps (e.g., RBG channels, or the like) resulting in pairs of images. Logic flow 700 can continue to block 706 where the pairs of images are used as input to a machine learning models 428 (e.g., NN, FCN, CNN, or the like) and the inference from the models 428 comprises an indication of the updated lumen border 426.

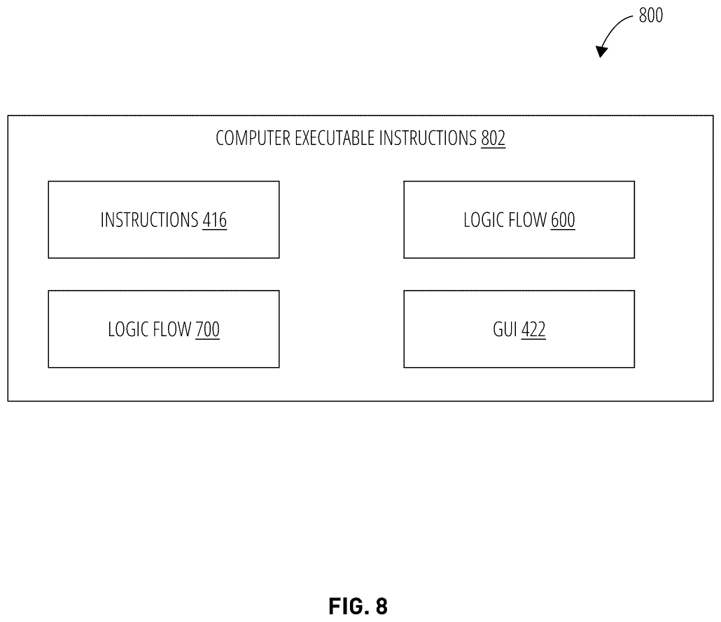

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., processor 106, processor 406, IVUS imaging system acquisition circuitry 414, and the like) can execute. For example, computer executable instructions 802 can include instructions to implement operations described with respect to instructions 416, logic flow 600, logic flow 700, and/or GUI 422. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

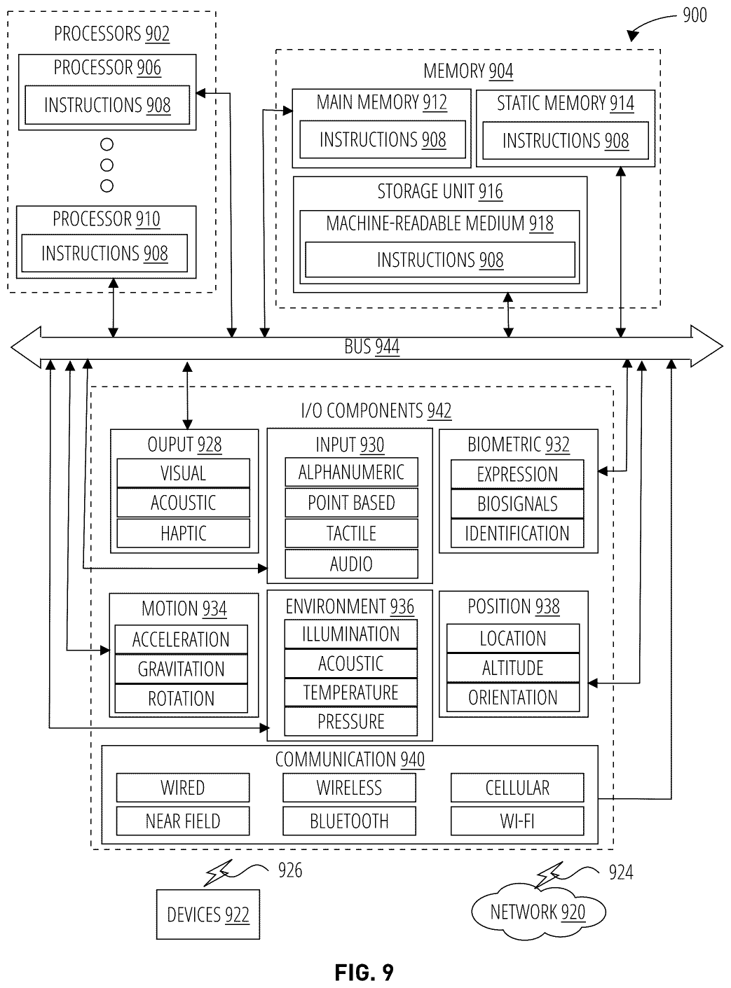

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to instructions 416 of FIG. 4, logic flow 600 of FIG. 6, logic flow 700 of FIG. 7, or the like. More generally, the instructions 908 may cause the machine 900 to identify an updated lumen border as described herein during a pre-PCI, peri-PCI, or post-PCI using IVUS. It is noted that the present disclosure provides specific and discrete implementations of GUI representations and behavior that is a significant improvement over the prior art. In particular, the present disclosure provides an improvement to computing technology in that lumen borders can be corrected without the time consuming requirement that a physician manually adjust each segment of the border.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while only a single machine 900 is illustrated, the term “machine” shall also be taken to include a collection of machines 900 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor's cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store, encoding, or carrying the instructions 908 for execution by the machine 900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A computer implemented method, comprising:
receiving, via an input device, an indication of a region of an intravascular ultrasound (IVUS) image of a vessel of a patient corresponding to a location either inside or outside a lumen border of the vessel;
generating, at a processing component of an IVUS system, an updated lumen border based on an initially detected lumen border and the region;
generating a graphical user interface (GUI) comprising visualizations of the IVUS image and the updated lumen border; and
causing the GUI to be displayed on a display.

2. The computer implemented method of claim 1, comprising receiving a series of IVUS images of the vessel of a patient from an IVUS catheter, the series of IVUS images comprising a plurality of frames, the IVUS image a one of the plurality of frames.

3. The computer implemented method of claim 1, comprising automatically detecting the initially detected lumen border based on the IVUS image.

4. The computer implemented method of claim 3, comprising:
generating an initial GUI comprising visualizations of the IVUS image and the initially detected lumen border; and
causing the initial GUI to be displayed on a display.

5. The computer implemented method of claim 4, wherein receiving the indication of the region of the IVUS image of the vessel comprises receiving via a mouse or a touch screen an indication of a location on the GUI corresponding to the region.

6. The computer implemented method of claim 1, wherein the region comprises an indication of whether the region is within or without the lumen border.

7. The computer implemented method of claim 6, wherein generating the updated lumen border comprises:
deriving a graph cut segmentation of the IVUS image; and
identifying the updated lumen border based on the graph cut segmentation and an iterative energy minimization where the region is the minimization parameter.

8. The computer implemented method of claim 6, wherein generating the updated lumen border comprises:
deriving a ranked list of a plurality of segmentation hypotheses; and
identifying the updated lumen border based on the ranked list of the plurality of segmentation hypotheses and the region.

9. The computer implemented method of claim 6, wherein generating the updated lumen border comprises:
concatenating a Euclidian distance map of the IVUS image and the region to generate a pair of images;
generating the updated lumen border based on an inference from a machine learning model using the pair of images as input to the machine learning model.

10. The computer implemented method of claim 9, wherein the machine learning model is a neural network.

11. The computer implemented method of claim 9, wherein the Euclidian distance map is based on a red, green, blue (RBG) channel segregation of the IVUS images and the region resulting in a plurality of pairs of images, and wherein the region is a first region, the method comprising:
receiving, via the input device, an indication of a second region of the IVUS image of the vessel of the patient corresponding to a location either within or without the lumen border of the vessel,
wherein the Euclidian distance map is based on the first region and the second region resulting in a plurality of pairs of images.

12. The computer implemented method of claim 11, wherein the first region is inside the lumen border and the second region is outside the lumen border.

13. An apparatus for an intravascular ultrasound (IVUS) imaging system, comprising:
a display;
a computer input device;
a processor coupled to the computer input device and the display; and
a memory coupled to the processor, the memory comprising instructions executable by the processor, which when executed cause the processor to:
receive, via the computer input device, an indication of a region of an IVUS image of a vessel of a patient corresponding to a location either inside or outside a lumen border of the vessel;
generate an updated lumen border based on an initially detected lumen border and the region;
generate a graphical user interface (GUI) comprising visualizations of the IVUS image and the updated lumen border; and
cause the GUI to be displayed on the display.

14. The apparatus of claim 13, wherein the region comprises an indication of whether the region is within or without the lumen border.

15. The apparatus of claim 14, the instructions, which when executed by the processor further cause the processor to:
derive a graph cut segmentation of the IVUS image; and
identify the updated lumen border based on the graph cut segmentation and an iterative energy minimization where the region is the minimization parameter.

16. The apparatus of claim 14, the instructions, which when executed by the processor further cause the processor to:
derive a ranked list of a plurality of segmentation hypotheses; and
identify the updated lumen border based on the ranked list of the plurality of segmentation hypotheses and the region.

17. The apparatus of claim 14, the instructions, which when executed by the processor further cause the processor to:
concatenate a Euclidian distance map of the IVUS image and the region to generate a pair of images; and
generate the updated lumen border based on an inference from a machine learning model using the pair of images as input to the machine learning model.

18. At least one machine readable storage device, comprising a plurality of instructions that in response to being executed by a processor of an intravascular ultrasound (IVUS) imaging system cause the processor to:
receive, via the computer input device coupled to the processor, an indication of a region of an IVUS image of a vessel of a patient corresponding to a location either inside or outside a lumen border of the vessel;
generate an updated lumen border based on an initially detected lumen border and the region;
generate a graphical user interface (GUI) comprising visualizations of the IVUS image and the updated lumen border; and
cause the GUI to be displayed on a display coupled to the processor.

19. The at least one machine readable storage device of claim 18, wherein the Euclidian distance map is based on a red, green, blue (RBG) channel segregation of the IVUS images and the region resulting in a plurality of pairs of images, and wherein the region is a first region, the instructions in response to be executed by the processor, further cause the processor to:
receive, via the input device, an indication of a second region of the IVUS image of the vessel of the patient corresponding to a location either within or without the lumen border of the vessel, wherein the Euclidian distance map is based on the first region and the second region resulting in a plurality of pairs of images.

20. The at least one machine readable storage device of claim 19, wherein the first region is inside the lumen border and the second region is outside the lumen border.
